(19) United States
(12) Patent Application Publication
(10) Pub. No.: US 2026/0207294 A1
(43) Pub. Date: Jul. 23, 2026

(54) CLEANING-FACILITATED SINGLE-OPENING CONTAINER

(71) Applicant: Dongguan Haohui Biotechnology Co., Ltd., Dongguan (CN)

(72) Inventor: Hao Huang, Dongguan (CN)

(21) Appl. No.: 19/569,237

(22) Filed: Mar. 17, 2026

(30) Foreign Application Priority Data
Mar. 21, 2025 (CN) 202520509881.3

(51) Int. Cl. A61B 90/70; A61B 10/00

(52) U.S. Cl. CPC A61B 90/70; A61B 10/0058

(57) ABSTRACT

A cleaning-facilitated single-opening container is provided, including a container body. One end of the container body is configured as an accessory end and is provided with a functional accessory, and another end is configured as an opening end. A plugging port is provided in the opening end, and a plugging portion is formed. The plugging portion has an accommodating space extending from the plugging port into the container body by a depth. A water outlet is formed in an end of the accommodating space. A water inlet is formed in the accessory end of the container body. The water inlet and the water outlet form pipeline communication, so that cleaning liquid flows in from the water inlet and flows out from the water outlet, to clean the accommodating space. In summary, cleaning efficiency and effect are enhanced through a unique design of the water inlet and the water outlet.

### TECHNICAL FIELD

The present disclosure relates to the technical field of semen collection equipment, and in particular, to a cleaning-facilitated single-opening container.

### BACKGROUND

In application scenarios of single-opening containers such as semen collection equipment, there are many problems that need to be urgently solved in the existing technology. To increase the collection speed and improve the comfort of a patient, a flexible material such as a silicone sleeve is often used to manufacture an accommodating silicone sleeve to assist in semen extraction. For some insensitive patients, a functional accessory such as a vibration accessory or a heating accessory needs to be mounted at one end of the equipment. During use, a lubricating material will be applied to a plugging portion of the other end of the equipment to reduce the pain of the patient due to plugging. However, it is a great challenge to clean the plugging portion after use. The plugging portion has a small diameter and a large depth, making it difficult to clean the plugging portion directly from a plugging port. It is also difficult to comprehensively and thoroughly clean an internal accommodating space. This cannot meet a hygiene requirement and may easily lead to potential risks such as cross infection. This has become a clear shortcoming of the existing technology.

For example, the Chinese patent document No. CN119074067A discloses a semen collection device and method for reproductive medicine, and relates to the technical field of sperm collection equipment. The device includes a semen collection device host. A silicone sleeve is movably disposed inside the semen collection device host, and a control panel is fixedly mounted on a top surface of the semen collection device host. Armrest frames are fixedly mounted on outer walls of two sides of the semen collection device host, and a mounting table is fixedly mounted on a bottom surface of the semen collection device host. A lifting mechanism is arranged on a bottom surface of the mounting table. The lifting mechanism includes four lifting rods and outer sleeve rods. The four lifting rods are fixedly connected to four corners of the bottom surface of the mounting table. The lifting rods are slidably disposed within the outer sleeve rods. A bottom surface of each outer sleeve rod is fixedly connected to a bracket. Universal wheels are mounted on the brackets, and adjustment mechanisms are arranged on the brackets.

In the above patent documents, there is no specific description on how to clean or clear the silicone sleeve after use. Apparently, the above problems also exist in practical use, so it is necessary to make improvements.

### SUMMARY

In order to overcome the drawbacks described above, the present disclosure aims to provide a technical solution capable of solving the above problems.

The present disclosure provides a cleaning-facilitated single-opening container, including a container body. One end of the container body is configured as an accessory end and is provided with a functional accessory. Another end of the container body is configured as an opening end. A plugging port is provided in the opening end, and a plugging portion is formed. The plugging portion has an accommodating space extending from the plugging port into the container body by a depth. A water outlet is formed in an end of the accommodating space. A water inlet is formed in the accessory end of the container body. The water inlet and the water outlet form pipeline communication, so that cleaning liquid flows in from the water inlet and flows out from the water outlet, to clean the accommodating space.

Further, the functional accessory is provided with a tail cover. The tail cover is mounted at the accessory end of the container body and is provided with a water inlet connector. One end of the water inlet connector is left on an outer side of the tail cover to form a free end. Another end of the water inlet connector extends into the container body and forms pipeline communication with the water outlet.

Further, the container body is further provided with an end protrusion at an end of the accommodating space. The end protrusion extends to the accessory end by a length. The end protrusion is internally hollowed to form a fluid channel. One end of the fluid channel is communicated with the water outlet, and another end of the fluid channel forms pipeline communication with the water inlet connector.

Further, the end protrusion is provided with a check device. One end of the check device is plugged into the end protrusion, and another end of the check device forms pipeline communication with the water inlet connector.

Further, the check device is provided with a short plug connector, a one-way valve body, and a long plug connector that are in pipeline communication with each other in sequence. One end of the long plug connector is abutted with the one-way valve body, and another end is plugged into the fluid channel inside the end protrusion. The one-way valve body is mounted between the short plug connector and the long plug connector, and prevents fluid from flowing from one end of the long plug connector to one end of the short plug connector. One end of the short plug connector is abutted with the one-way valve body, and another end of the short plug connector is in pipeline communication with the water inlet connector.

Further, a communication hose is disposed between the water inlet connector and the short plug connector. One end of the communication hose is sleeved on and forms fixed connection with the water inlet connector, and another end of the communication hose is sleeved on and forms fixed connection with the short plug connector, so that a pipeline communication state is maintained between the water inlet connector and the short plug connector.

Further, a component box is further disposed within the functional accessory. The component box is embedded and mounted at the accessory end of the container body to mount components.

Compared with the prior art, the present disclosure has the beneficial effects:

1. Since the water inlet is formed in the accessory end of the container body and the water outlet is formed in the end of the plugging portion, the cleaning liquid can flow in from the water inlet and flow out from the water outlet, to perform cleaning from the end of the accommodating space, so that dirty water is discharged from the plugging port. This manner significantly enhances cleaning efficiency and can quickly and conveniently clean the accommodating space. Meanwhile, the cleaning effect is significantly enhanced, and the interior of the accommodating space can be deeply cleaned to ensure comprehensive and complete cleaning. This effectively solves the problems that it is difficult for cleaning and cleaning is not thorough in the existing technology.

2. The functional accessory is provided with the tail cover and the water inlet connector to facilitate the introduction of an external water flow or the cleaning liquid into the container, thus further optimizing the cleaning process. The end protrusion arranged at the end of the accommodating space extends into the component box and is protected by a rigid shell of the component box. This prevents easy deformation due to the flexible material used to manufacture the container body. This affects connection to and even disconnection from the a cleaning pipeline, and ensures the stability of the cleaning system.

3. The arrangement of the check device effectively prevents the dirty water from flowing back and contaminating a pipeline, thus enhancing the hygiene and safety of the equipment. The check device adopts a structure including the short plug connector, the one-way valve body, and the long plug connector that are communicated with each other in sequence. The elongated design of the long plug connector enhances the firmness and stability of the connection with the end protrusion, thus avoiding disconnection caused by a soft material of the container body.

Through the foregoing improvements, the present disclosure can provide the cleaning-facilitated single-opening container. The unique water inlet and water outlet design enhances the cleaning efficiency and effect. Stability of a cleaning system is ensured through optimization structures such as the tail cover, the communication hose, and the end protrusion. The check device prevents the dirty water from flowing back, thus comprehensively solving the problem of cleaning of the single-opening container and enhancing the hygiene and the safety.

The additional aspects and advantages of the present disclosure will be partially provided in the following descriptions, some of which will become apparent from the following descriptions, or learned through the practice of the present disclosure.

### BRIEF DESCRIPTION OF THE DRAWINGS

To describe the technical solutions in the embodiments of the present disclosure or in the prior art more clearly, the following briefly introduces the accompanying drawings for describing the embodiments or the related art. Apparently, the accompanying drawings in the following description show merely some embodiments of the present disclosure, and a person of ordinary skill in the art may still derive other drawings from the accompanying drawings without creative efforts.

FIG. 1 is a schematic diagram of a cross section of a container body of the present disclosure;

FIG. 2 is a schematic diagram of a cross section of an accommodating space and a check device of the present disclosure;

FIG. 3 is a schematic diagram of a structure of a container body and a tail cover of the present disclosure;

FIG. 4 is a schematic diagram of a cross section of a check device and a tail cover of the present disclosure;

FIG. 5 is a schematic diagram of a structure of a separated state of a container body and a component box of the present disclosure; and

FIG. 6 is a schematic diagram of a structure of a separated state of a check device of the present disclosure.

Reference numerals and names in the drawings are as follows:

10: container body; 11: accessory end; 12: groove; 13: water inlet; 20: opening end; 21: plugging port; 22: plugging portion; 30: accommodating space; 31: water outlet; 32: end protrusion; 33: check device; 34: short plug connector; 35: one-way valve body; 36: long plug connector; 40: component box; 41: through hole; 42: tail cover; 43: water inlet connector; and 44: communication hose.

### DETAILED DESCRIPTION OF THE EMBODIMENTS

The technical solutions in the embodiments of the present disclosure are clearly and completely described below. Apparently, the described embodiments are merely some embodiments of the present disclosure, rather than all of the embodiments. All other embodiments obtained by a person of ordinary skill in the art based on the embodiments of the present disclosure without making creative efforts shall fall within the protection scope of the present disclosure.

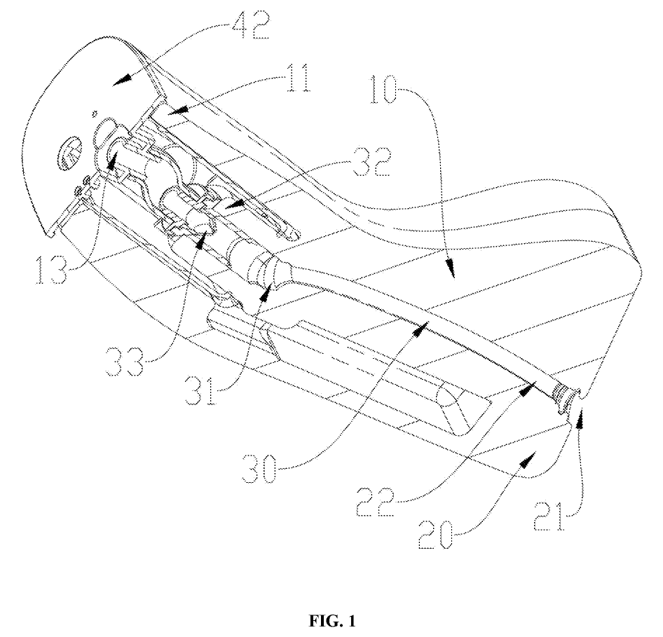

Referring to FIG. 1 to FIG. 6, in the embodiments of the present disclosure, a cleaning-facilitated single-opening container includes a container body 10. One end of the container body 10 is configured as an accessory end 11 and is provided with a functional accessory. Another end of the container body 10 is configured as an opening end 20. A plugging port 21 is provided in the opening end 20, and a plugging portion 22 is formed. The plugging portion 22 has an accommodating space 30 extending from the plugging port 21 into the container body 10 by a depth. A water outlet 31 is formed in an end of the accommodating space 30. A water inlet 13 is formed in the accessory end 11 of the container body 10. The water inlet 13 and the water outlet 31 form pipeline communication, so that cleaning liquid flows in from the water inlet 13 and flows out from the water outlet 31, to clean the accommodating space 30.

Specifically, in some semen collection equipment, to increase the collection speed and optimize the comfort of a patient, an accommodating rubber sleeve made of a flexible material is usually provided, such as a silicone sleeve, to assist a patient to complete a semen extraction process. For some insensitive patients, a functional accessory achieving an assisting function further needs to be mounted in the equipment, such as a component with a vibration function or a component with a heating function. Usually, these functional accessories can be mounted at one end of the equipment away from a user, and the plugging portion 22 used by a patient is arranged at another end of the equipment close to the user. In a specific use process, a lubricating material will be applied into the plugging portion 22 to assist in lubrication and reduce the pain of the patient due to plugging. After use, the equipment or the plugging portion 22 of the container needs to be cleaned.

Then, since the plugging portion 22 has a relatively small diameter and a relatively large depth, it is relatively hard to clean the container from the plugging port 21, and it is not easy to perfectly clean the interior of the accommodating space 30 of the plugging portion 22. Therefore, it is necessary to make improvements.

Therefore, the water inlet 13 is formed in another end of the equipment or the container, and the water outlet 31 is formed in the end of the plugging portion 22. A water flow or other cleaning liquid can directly flow in through the water inlet 13 and flow out from the water outlet 31, to start cleaning from the end of the accommodating space 30, so that dirty water after cleaning can be directly discharged from the plugging port 21. This can be convenient for efficiently cleaning the accommodating space 30 and can also enhance the cleaning effect, so that the accommodating space 30 is perfectly cleaned.

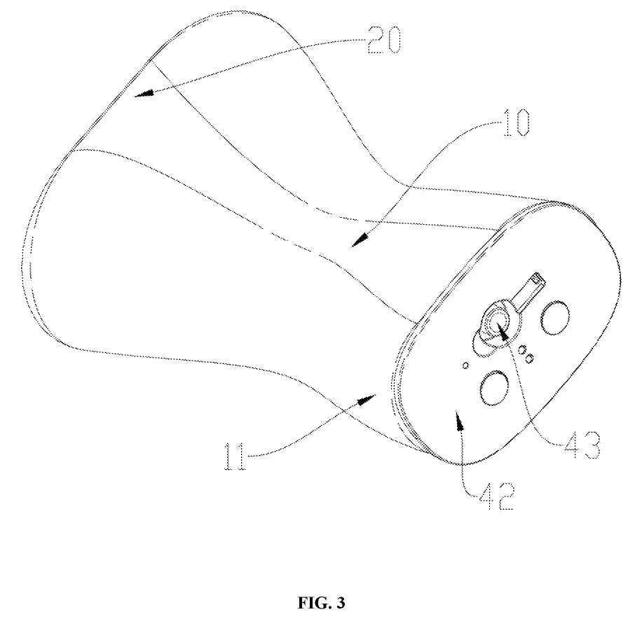

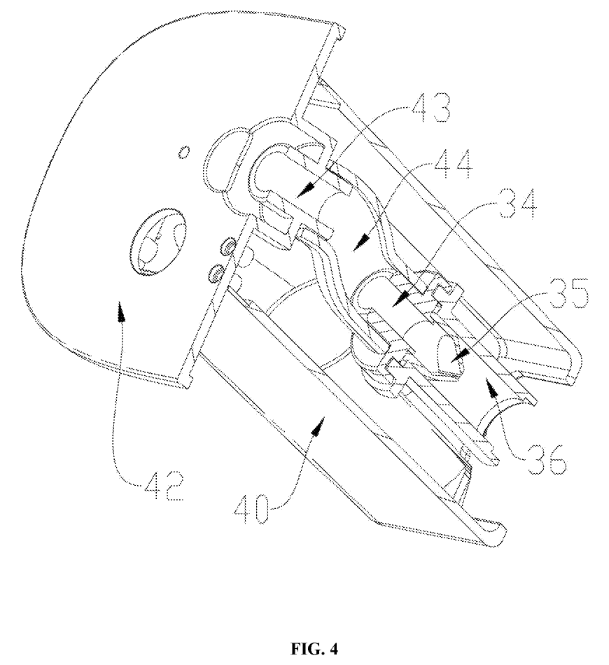

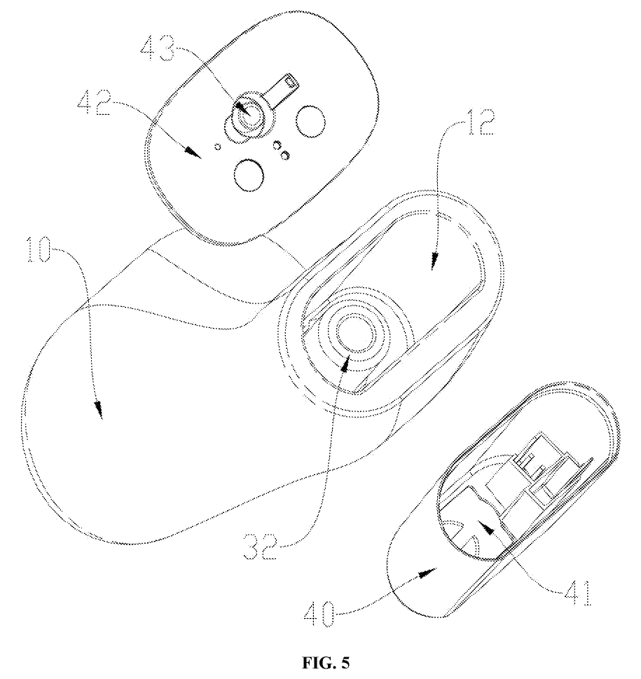

As shown in FIG. 1, FIG. 3, FIG. 4, and FIG. 5, preferably, the functional accessory is provided with a tail cover 42. The tail cover 42 is mounted at the accessory end 11 of the container body 10 and is provided with a water inlet connector 43. One end of the water inlet connector 43 is left on an outer side of the tail cover 42 to form a free end. Another end of the water inlet connector 43 extends into the container body 10 and forms pipeline communication with the water outlet 31.

Specifically, to mount a corresponding functional component, the accessory end 11 of the equipment or the container is usually provided with the tail cover 42 and a component box 40, and the corresponding functional component is mounted inside the component box 40. The tail cover 42 can be provided with the water inlet connector 43, so that an external water flow or cleaning liquid can be conveniently introduced into the container through the free end of the water inlet connector 43 and discharged from the water outlet 31, to clean the accommodating space 30.

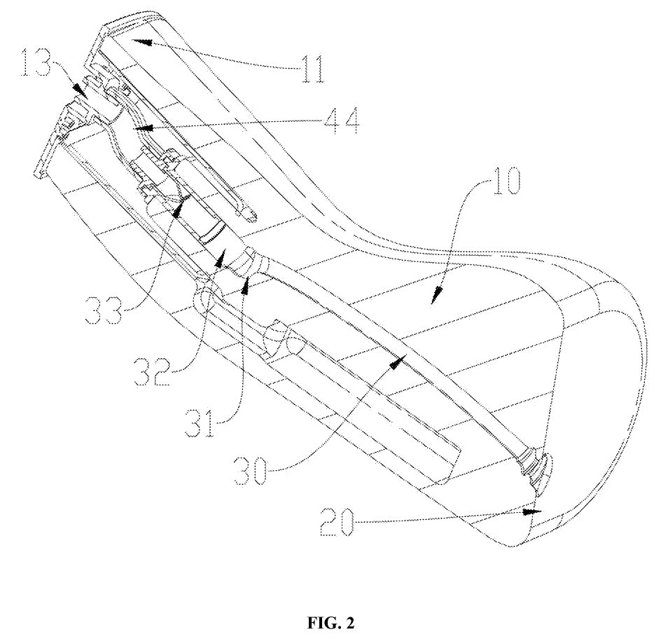

As shown in FIG. 1, FIG. 2, and FIG. 5, preferably, the container body 10 is further provided with an end protrusion 32 at an end of the accommodating space 30. The end protrusion 32 extends to the accessory end 11 by a length. The end protrusion 32 is internally hollowed to form a fluid channel. One end of the fluid channel is communicated with the water outlet 31, and another end of the fluid channel forms pipeline communication with the water inlet connector 43.

Specifically, since the container body 10 is usually made of a flexible material, the end protrusion 32 can be arranged at the end of the accommodating space 30 to facilitate connection with a corresponding water flow pipeline. The end protrusion 32 can extend into the component box 40 by a length, so that the end protrusion 32 is protected by using a relatively rigid shell of the component box 40, to prevent disconnection from a pipeline due to the deformation of the end protrusion 32.

As shown in FIG. 1 and FIG. 2, preferably, the end protrusion 32 is provided with a check device 33. One end of the check device 33 is plugged into the end protrusion 32, and another end of the check device 33 forms pipeline communication with the water inlet connector 43.

Specifically, to prevent dirty water from flowing back and contaminating the pipeline, the check device 33 can also be provided. The check device 33 is arranged at one end of the end protrusion 32 communicated with the water inlet connector 43. The check device 33 is configured to prevent liquid from flowing from the water outlet 31 to the water inlet 13.

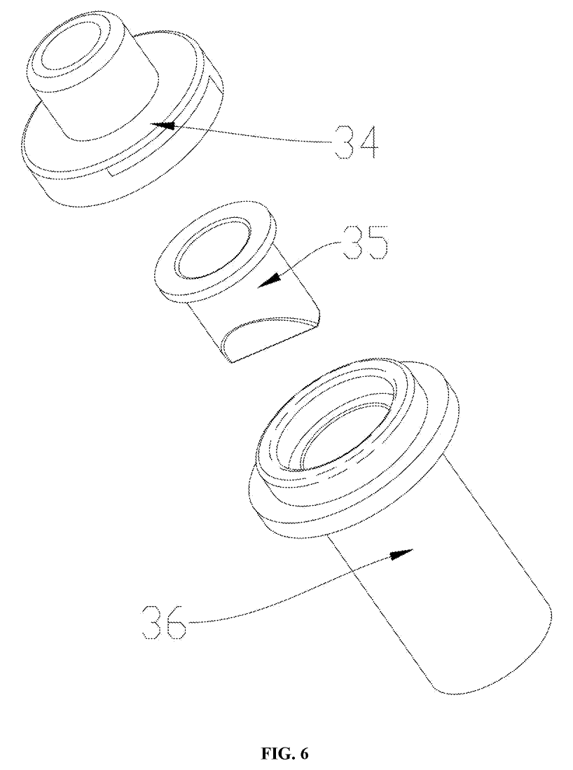

As shown in FIG. 4 and FIG. 6, preferably, the check device 33 is provided with a short plug connector 34, a one-way valve body 35, and a long plug connector 36 that are in pipeline communication with each other in sequence. One end of the long plug connector 36 is abutted with the one-way valve body 35, and another end is plugged into the fluid channel inside the end protrusion 32. The one-way valve body 35 is mounted between the short plug connector 34 and the long plug connector 36, and prevents fluid from flowing from one end of the long plug connector 36 to one end of the short plug connector 34. One end of the short plug connector 34 is abutted with the one-way valve body 35, and another end of the short plug connector 34 is in pipeline communication with the water inlet connector 43.

Specifically, due to the relatively soft material of the container body 10, such as a silicone material, it is prone to detachment when the container body is connected to a connector. Therefore, the extended long plug connector 36 can be provided. The relatively long plugging portion 22 can be plugged into the end protrusion 32 by a depth, to enhance the firmness and stability of the container body and prevent detachment.

Then, it is preferred that the one-way valve body 35 is a micro diaphragm type one-way valve without a spring structure, or a duckbill type one-way valve. The duckbill type one-way valve allows fluid to flow through the hose. An end of the hose has a natural flat shape. A water flow opens the duckbill type flat end, to allow a medium to pass through. When a pressure is removed from an inlet side, the duckbill end returns to its flat state, thereby cutting off the flow.

As shown in FIG. 2 and FIG. 4, preferably, a communication hose 44 is disposed between the water inlet connector 43 and the short plug connector 34. One end of the communication hose 44 is sleeved on and forms fixed connection with the water inlet connector 43, and another end of the communication hose 44 is sleeved on and forms fixed connection with the short plug connector 34, so that a pipeline communication state is maintained between the water inlet connector 43 and the short plug connector 34.

Specifically, to allow a water flow to flow normally from the water inlet connector 43 to the water outlet 31, preferably, the communication hose 44 with a particular length is provided to establish achieve pipeline communication between the water inlet connector 43 and the check device 33. Furthermore, the communication hose 44 can be made of a relatively long material with a particular flexibility, such as polyethylene (PE) hose and a silicone hose, so that two ends of the communication hose can be subjected to a pulling force to maintain a stable communication state.

As shown in FIG. 5, preferably, a component box 40 is further disposed within the functional accessory. The component box 40 is embedded and mounted at the accessory end 11 of the container body 10 to mount components.

Specifically, to facilitate the mounting of the functional component, preferably, the component box 40 is provided and is embedded and mounted into a groove 12 of the accessory end 11. To protect the components, preferably, the component box 40 and the tail cover 42 are made of relatively rigid materials, such as hard plastic and commonly known thermosetting plastic such as phenolic plastic, epoxy plastic, amino plastic, unsaturated polyester, and alkyd plastic. The tail cover 42 is preferably covered at an opening of the component box 40, and the communication hose 44 is disposed within the component box 40.

Then, a through hole 41 is further formed in a bottom of the component box 40, so that the end protrusion 32 can enter the component box 40 through the through hole 41, thereby facilitating the plugging of the long plug connector 36 of the check device 33 into the fluid channel inside the end protrusion 32 and forming fixed connection. Other corresponding functional components such as a vibrator, a heater, and a negative pressure generator can be mounted inside the component box 40 as needed, and corresponding buttons can be arranged on the tail cover 42 to control the functional components.

For those skilled in the art, it is apparent that the present disclosure is not limited to the details of the exemplary embodiments mentioned above, and can be implemented in other specific forms without departing from the spirit or basic features of the present disclosure. Therefore, in any perspective, the embodiments should be regarded as exemplary and non-restrictive. The scope of the present disclosure is limited by the accompanying claims rather than the above description. Therefore, all changes within the meaning and scope of the equivalent conditions of the claims within the present disclosure.

What is claimed is:

1. A cleaning-facilitated single-opening container, comprising a container body (10), wherein one end of the container body (10) is configured as an accessory end (11) and is provided with a functional accessory; another end of the container body (10) is configured as an opening end (20); a plugging port (21) is provided in the opening end (20), and a plugging portion (22) is formed; the plugging portion (22) has an accommodating space (30) extending from the plugging port (21) into the container body (10) by a depth; a water outlet (31) is formed in an end of the accommodating space (30); a water inlet (13) is formed in the accessory end (11) of the container body (10); the water inlet (13) and the water outlet (31) form pipeline communication, so that cleaning liquid flows in from the water inlet (13) and flows out from the water outlet (31), to clean the accommodating space (30).

2. The cleaning-facilitated single-opening container according to claim 1, wherein the functional accessory is provided with a tail cover (42); the tail cover (42) is mounted at the accessory end (11) of the container body (10) and is provided with a water inlet connector (43); one end of the water inlet connector (43) is left on an outer side of the tail cover (42) to form a free end; and another end of the water inlet connector (43) extends into the container body (10) and forms pipeline communication with the water outlet (31).

3. The cleaning-facilitated single-opening container according to claim 2, wherein the container body (10) is further provided with an end protrusion (32) at an end of the accommodating space (30); the end protrusion (32) extends to the accessory end (11) by a length; the end protrusion (32) is internally hollowed to form a fluid channel; and one end of the fluid channel is communicated with the water outlet (31), and another end of the fluid channel forms pipeline communication with the water inlet connector (43).

4. The cleaning-facilitated single-opening container according to claim 3, wherein the end protrusion (32) is provided with a check device (33); and one end of the check device (33) is plugged into the end protrusion (32), and another end of the check device (33) forms pipeline communication with the water inlet connector (43).

5. The cleaning-facilitated single-opening container according to claim 4, wherein the check device (33) is provided with a short plug connector (34), a one-way valve body (35), and a long plug connector (36) that are in pipeline communication with each other in sequence; one end of the long plug connector (36) is abutted with the one-way valve body (35), and another end is plugged into the fluid channel inside the end protrusion (32); the one-way valve body (35) is mounted between the short plug connector (34) and the long plug connector (36), and prevents fluid from flowing from one end of the long plug connector (36) to one end of the short plug connector (34); and one end of the short plug connector (34) is abutted with the one-way valve body (35), and another end of the short plug connector (34) is in pipeline communication with the water inlet connector (43).

6. The cleaning-facilitated single-opening container according to claim 5, wherein a communication hose (44) is disposed between the water inlet connector (43) and the short plug connector (34); and one end of the communication hose (44) is sleeved on and forms fixed connection with the water inlet connector (43), and another end of the communication hose (44) is sleeved on and forms fixed connection with the short plug connector (34), so that a pipeline communication state is maintained between the water inlet connector (43) and the short plug connector (34).

7. The cleaning-facilitated single-opening container according to claim 1, wherein a component box (40) is further disposed within the functional accessory; and the component box (40) is embedded and mounted at the accessory end (11) of the container body (10) to mount components.
